(19) United States
(12) Patent Application Publication
(10) Pub. No.: US 2017/0124266 A1
(43) Pub. Date: May 4, 2017

(54) Method for Practitioner Network and Appointment Management System

(71) Applicants: Muhammad Ali Hasan, Huntington Beach, CA (US); David MacLeod, Denver, CO (US)

(72) Inventors: Muhammad Ali Hasan, Huntington Beach, CA (US); David MacLeod, Denver, CO (US)

(21) Appl. No.: 15/342,051

(22) Filed: Nov. 2, 2016

Related U.S. Application Data
(60) Provisional application No. 62/250,358, filed on Nov. 3, 2015.

(51) Int. Cl. G06F 19/00; G06F 3/0482

(52) U.S. Cl. CPC G06F 19/327; G06F 19/322; G06F 3/0482

(57) ABSTRACT

A practitioner network and appointment management system is presented that coordinates between end users and practitioners to enable networked appointment scheduling and management. The system includes an end user side and a practitioner side. End user side enables control of a scalable interactive map whereon practitioner data points are displayable by medical discipline. Practitioner first order data (including time to availability) is visually cued. Selection of a practitioner data point enables end user entry to a practitioner queue, which updates practitioner first order data across the network. Patient data is automatically transferrable to the practitioner queue by secure protocol. Practitioner side enables access of practitioner queue and selection of patients occupying designated slots therein to view associated patient data. Patient locations are displayable to practitioner for in-person visits. Practitioners can transfer patients into other practitioner queues. HIPPA compliant secure data exchange and financial transactions are enabled across the network.

### CROSS-REFERENCE TO RELATED APPLICATIONS

This nonprovisional application for utility patent claims the benefit of provisional application No. 62/250,358 filed on Nov. 3, 2015

### FEDERALLY SPONSORED RESEARCH OR DEVELOPMENT

Not Applicable

### INCORPORATION BY REFERENCE OF MATERIAL SUBMITTED ON A COMPACT DISK

Not Applicable

### COPYRIGHT NOTICE

Some portions of the disclosure of this patent document may contain material subject to copyright protection. The copyright owner has no objection to the facsimile reproduction by anyone of the patent document or ensuing disclosure as it appears on record at the Patent and Trademark Office, but otherwise reserves all copyright rights whatsoever.

### BACKGROUND OF THE INVENTION

Various networks exist able to coordinate between multiple users in space and time. But few are directed to coordinating patients and medical practitioners whereby wait time for any particular patient to see any particular practitioner is minimized. The present method for practitioner network and appointment management system, therefore, has been devised to enable end users expedient determination of availability of practitioners of desired specialties and disciplines and then effect appointment scheduling as preferred by action coordinated through peripheral devices participating in a secure network by means of the present method.

The present method for practitioner network and appointment management system includes an end user side and a practitioner side controllable through a Graphical User Interface (“GUI”) operable upon a peripheral computing device, or Wireless Communications Device (“WCD”). End users are enabled to locate desired practitioners upon an interactive map viewable upon the WCD, whereon practitioner data points display visibly significant of availability, specialty or discipline, time to availability, and proximity to the end user's current location, among additional or other first order information. Selection of a desired practitioner automates entry of end user data input into an appointment schedule whereby said end user data is automatically enterable into the relevant practitioner's queue. The practitioner's data point is thereby updated across all users participating in the network to reflect entry of the end user data into the practitioner's queue. Thus availability, and time to availability, of the particular practitioner is updated in real time, and end user confidential data is captured by the practitioner for reference.

The present system is coordinated with a Global Positioning System (“GPS”) whereby users are routable and directable to each other, when desired. Thus practitioners may select patients displayable in their queue to visit, and patients may travel to selected practitioners whose queues they have entered.

Patient data, automated into a practitioner queue when an end user selects a desired practitioner, may include additional biometric data, medical data, medical history data, current insurance information, or other data, as made accessible via operational communication with a third party, or enabled downloadable from an end user account storable upon a central server or cached in WCD memory.

Thus the present method for practitioner network and appointment management system expedites scheduling of medical appointments to minimize a patient's wait time for a medical professional in a desired discipline or field of practice.

### FIELD OF THE INVENTION

The present invention relates to a method for practitioner network and appointment management system, and more particularly, to a method for practitioner network and appointment management system that minimizes a patient's wait time to see a desired medical professional by coordinating a network of practitioners and end users whereby said end users may display practitioners of a selected discipline upon an interactive map, expeditiously perceive availability of said practitioners, and effect an appointment scheduling in relation to said availability, discipline, and proximity of the practitioner to a current location of the end user.

Practitioners are enabled to view queues of patients scheduled for appointment, capture relevant data (including biometric data, insurance information, medical history or other data provisional from a third party data source integrated with, or accessible by, the present network) automatically entered into the queue when an end user selects the practitioner for appointment, transfer or refer patients to other practitioners participating in the network, and gain directions to a particular patient to whom an in person visit is preferable.

The present method for practitioner network and appointment management system, therefore, enables coordination between patients and care providers to better enable efficient dispatch of medical treatment while empowering end users to locate and reach particular medical practitioners expeditiously, as desired.

### SUMMARY OF THE INVENTION

The general purpose of the method for practitioner network and appointment management system, described subsequently in greater detail, is to provide a method for practitioner network and appointment management system which has many novel features that result in a method for practitioner network and appointment management system which is not anticipated, rendered obvious, suggested, or even implied by prior art, either alone or in combination thereof.

The present method for practitioner network and appointment management system has been devised to network practitioners and patients together into a shared user space whereby appointment scheduling is expediently dispatched and patients and practitioners are readily matched according to proximity, specialty, and availability, as preferred. Additionally, billing, payment, and insurance concerns are expediently dispatched between networked peers.

The Internet age has enabled new methods of coordinating between users, and more and more practitioners are now able to make house calls and meet with patients according to their own convenience and schedules. The present method increases this convenience by networking practitioners together whereby patients are expediently transferable between practitioners, and practitioners are readily selectable by patients. Thus patient time to practitioner availability is minimized.

The present method for practitioner network and appointment management system, therefore, is operable upon a peripheral computing device, or Wireless Communications Device (“WCD”), participating in network with a central server. Data is transferable across the network, over secure Internet protocol, to effect updates to all peripherals or WCDs participating in the network in real time.

The instant method for practitioner network and appointment management system presents a Graphic User Interface (“GUI”) for interaction with a user whereby selection and data transfer between users is effective. The GUI presents as a home screen including a Global Positioning System (“GPS”) enabled interactive map whereon user position is displayable.

In the present disclosure, users are split into two groups—the end user (i.e. patient seeking a practitioner) and the practitioner (i.e. a medical professional practicing in a particular specialty or discipline). For end user operators of the present method, selection of a practitioner type menu prompt enables filtering of practitioner data points displayable in relevant location upon the interactive map, whereby particular practitioners of a selected discipline are ascertainable for appointment scheduling. Example disciplines or specialties (or other medical categories) include, for example, dermatologists, radiologists, oncologists, physical therapists, chiropractors, pediatrics, psychologists, and hypnotherapists among other disciplines, fields, and specialties, as case may be, limited only by the number of practitioners participating in the network. New specialties may be added into the network by a particular practitioner entering the network, such categories updateable through a “settings” link enabling data input through a practitioner side GUI.

The interactive map is scalable, whereby practitioner's proximity to end user current location is displayable at a desired range. Time to reach each practitioner is displayed as part of first order information displayable with each practitioner data point, as will be described subsequently.

Practitioner data points display with visual cues indicative of availability. Unavailable practitioners are visually demarked relative available practitioners. In the present example herein illustrated, practitioner data points are articulated as flags signaled as either white or black. White flags signal availability of practitioner, wherein first order information is displayable in the flag (or when a user hovers a cursor over the flag or selects the flag through a touchscreen operable upon a WCD, as case may be), and black flags signal unavailability of a practitioner. Additional or other visual cues are contemplated as part of this invention, the present examples included merely illustrative of the principles of operation of the present method upon a WCD.

First order information is initial data displayed at any practitioner data point. In the present example, first order information includes the practitioner name, field of expertise (specialty or discipline), the number of patients already entered into the practitioner's queue, time to availability (how long until the first available appointment in the practitioner's queue) and the distance and/or travel time required to arrive at the practitioner. Additional information may be articulated and displayed as first order information, as case may be.

An end user may select any one of the practitioner data points and enter into that particular practitioner's queue, filling an available slot in the practitioner's appointment schedule. This process is considered expeditious, wherein the number of steps to complete entry into an available appointment slot is minimized. In its simplest form, then, an end user operating the method may simply select a desired practitioner, based on availability, discipline, and proximity, and then confirm the desired appointment. Relevant patient data is then transferred into the practitioner's queue automatically for display on the practitioner's side of the network, and the first order information displayable over the interactive map is updated for all users (i.e. the number of patients listed in the practitioner's queue and the time to availability is updated appropriately at the practitioner's data point as displayed across all users participating in the network). Practitioner may nonetheless receive confidential information effected when the appointment is scheduled, such confidential information including, for example, insurance information, patient biometric data, patient history, and additional medical data potentially storable and accessible through a third party platform sharable with, or accessible by, the present method. The purpose being to expedite patient intake by automated population of patient information into the practitioner's side of the network. The end user is then entered into the first available, or preferentially selected, time slot in the practitioner appointment schedule, and queued appropriate thereto.

The present method for practitioner network and appointment management system further enables Global Positioning of users, whereby operational communication with a Global Positioning System (“GPS”) enables routing of users to destinations selectable from other user positions. Thus an end user may be directed to a particular, selected practitioner, and a practitioner may likewise be directed to a particular end user current (or upcoming) in said practitioner's queue.

Practitioners may transfer patients to other practitioners through the practitioner side of the network. Practitioner side GUI of the network displays an interactive map whereon practitioner can filter additional practitioners or reveal patient locations. Practitioner is enabled administration and management of practitioner's queue, and may display the queue by selection inside a practitioner home screen. Patients are listed and selectable in the queue. Patient first order information may be displayable in the queue or when patients are selected. Selection of patient to display first order, or second order, information may be effected by enhanced selection (such as, for example, right-click or control click with a computer-aided display control, shift click, or by specific touch upon a touchscreen).

The practitioner queue may further list a set of actions associated with each patient, such as “complete” link, “transfer” link, and “go” link. In this example embodiment, “complete” link removes the patient from the practitioner queue and updates patient number and time to availability data displayable across all users participating in the network; “transfer” link enables selection of another practitioner, displayable upon the interactive map, and positions the patient into said another practitioner's queue when selected; and “go” link enters GPS mode to route and direct the practitioner to the patient's current (or last known) location as signaled through the network in real time.

Verification of practitioner licenses, or other means to verify lawful practice, may be required before a practitioner can enter the network. Practitioners may display as “qualified” or “verified” users where practitioners may optionally provide verification of licensing, or other qualifications, subsequent entry to the network. The present method may interface with licensing databases and determine practitioner registration verifiable though third-party systems before allowing entry into the network

The present method for practitioner network and appointment management system therefore enables expeditious management of practitioner appointments and minimizes patient wait time to see a qualified practitioner.

The present method for practitioner network and appointment management system further enables expedited payment and transfer of relevant insurance information between patients and practitioners and may handle payment solutions directly between parties when an appointment has been completed. Moreover, searching between practitioners who accept certain or particular insurance carriers over others is also contemplated as effective and displayable upon the interactive map.

Secure internet protocol preserves confidentiality of all data transmissions and novel file formatting may require use of a particular, registered software application able to unscramble unique data streams. Further, cryptographic keys may be used to privatize data streams within secure sockets identifiable only by public and private key sharing between users. Additional means of effecting secure Internet protocol and data transmission over network are contemplated as part of this invention.

Thus has been broadly outlined the more important features of the present method for practitioner network and appointment management system so that the detailed description thereof that follows may be better understood and in order that the present contribution to the art may be better appreciated.

Objects of the present method for practitioner network and appointment management system, along with various novel features that characterize the invention are particularly pointed out in the claims forming a part of this disclosure. For better understanding of the method for practitioner network and appointment management system, its operating advantages and specific objects attained by its uses, refer to the accompanying drawings and description.

### BRIEF DESCRIPTION OF THE DRAWINGS

### Figures

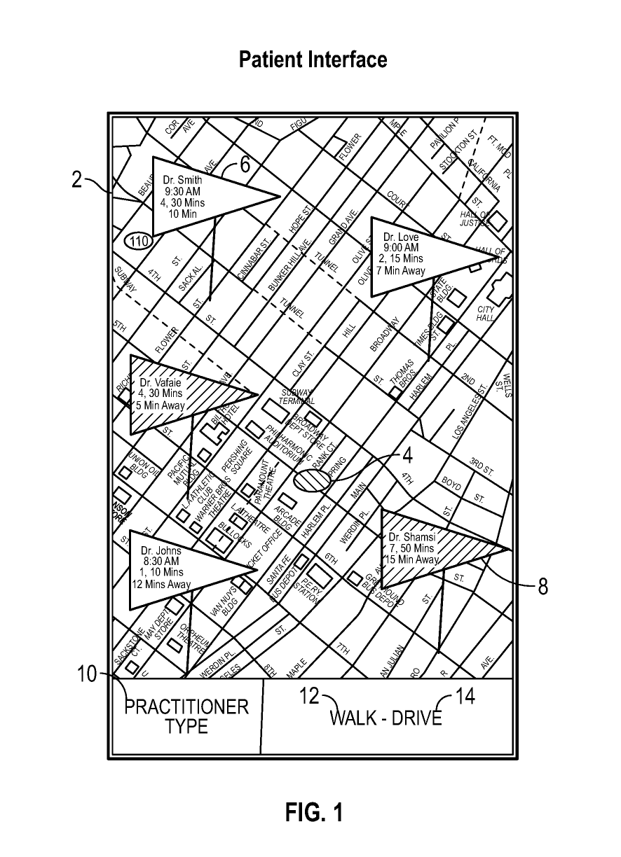

FIG. 1 is an example embodiment of a screen shot showing a home screen with interactive map displayable on an end user WCD.

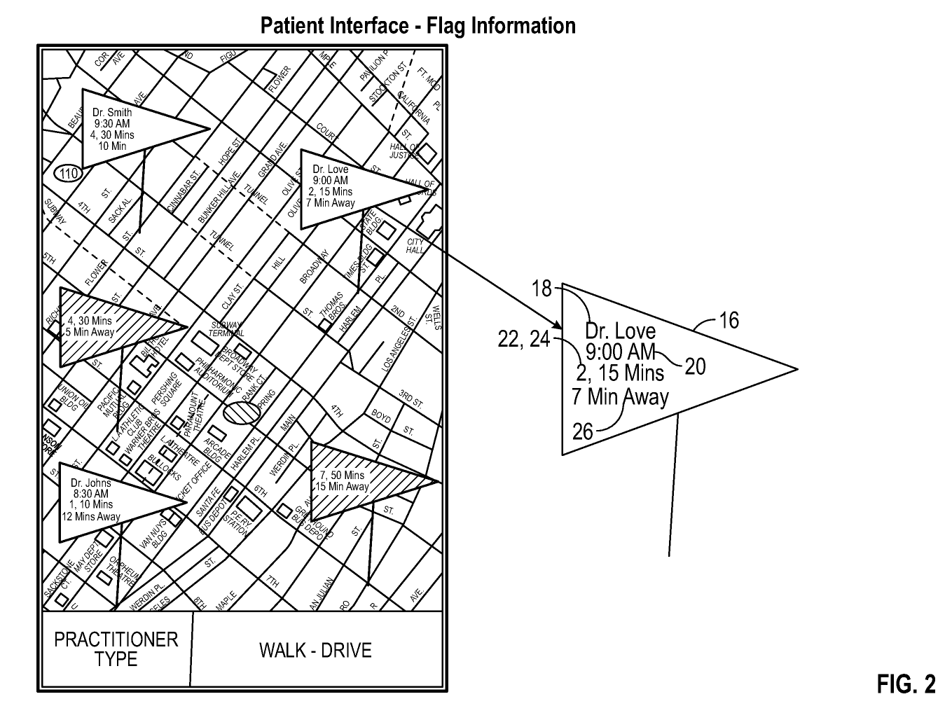

FIG. 2 is an example embodiment of a screen shot showing first order information displayable interior to a practitioner data point as displayed on an end user WCD.

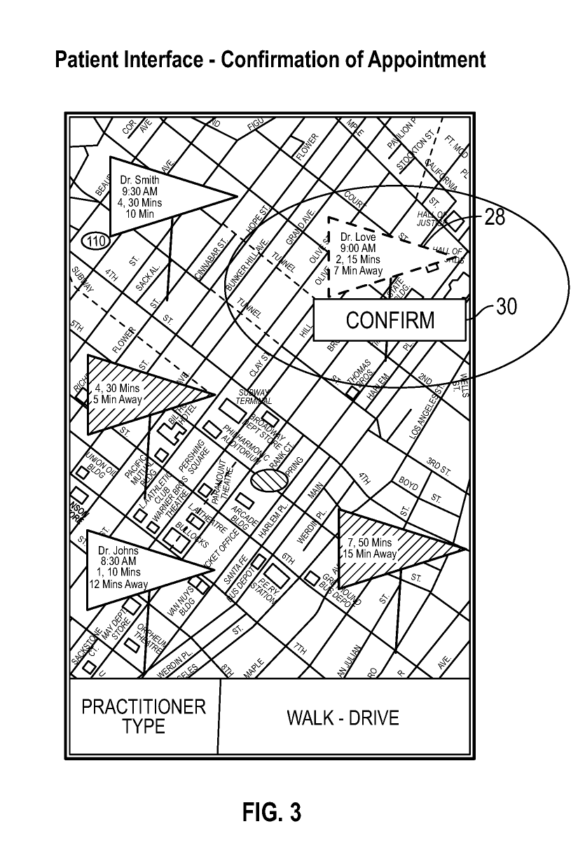

FIG. 3 is an example embodiment of a screen shot showing a confirmation prompt for an end user to enter patient data and join a particular practitioner's queue.

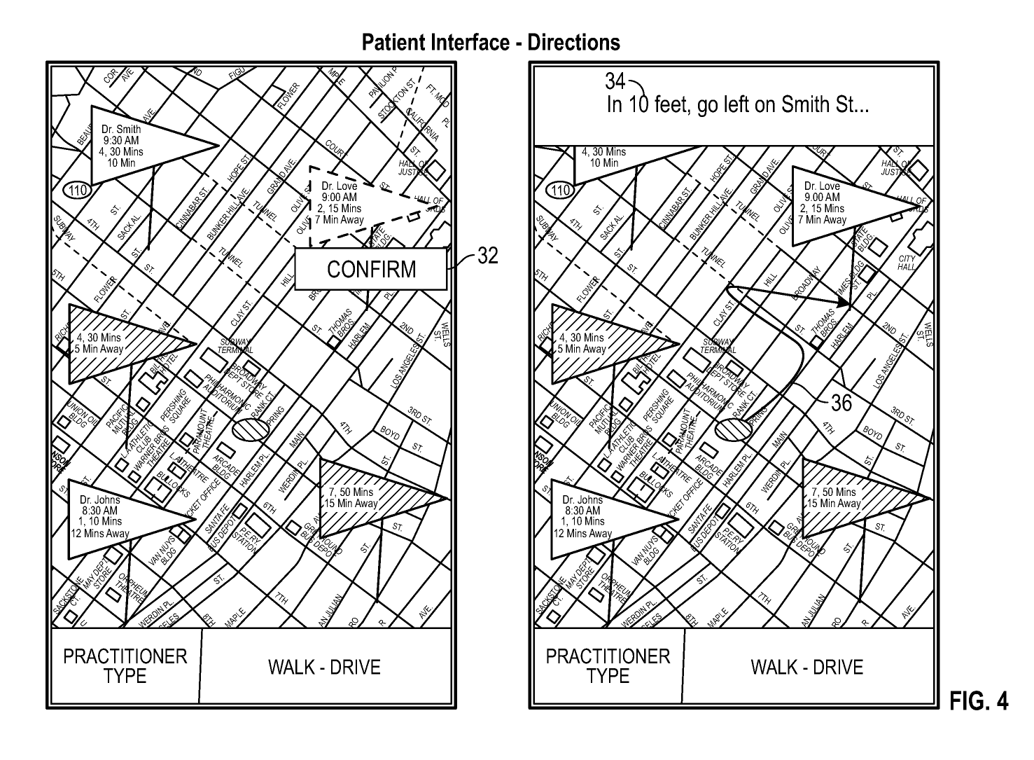

FIG. 4 is an example embodiment of a screen shot showing operation of a GPS mode to route and direct an end user, from a sensed current location, to the location of a selected practitioner.

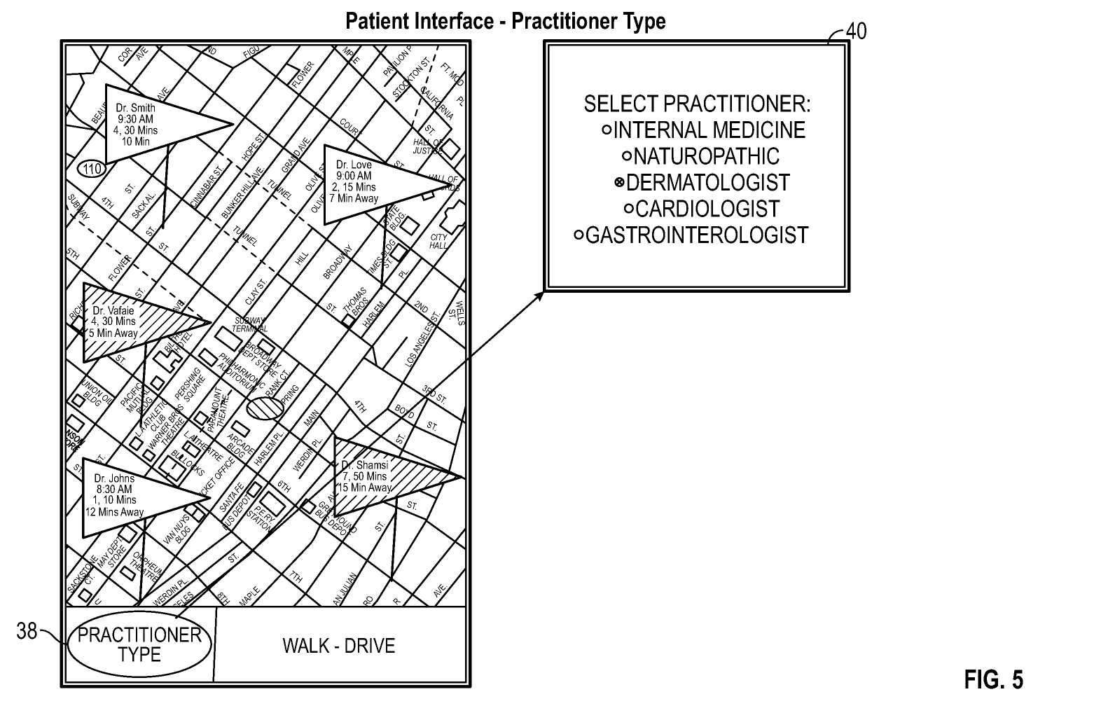

FIG. 5 is an example embodiment of a screen shot showing selection of a practitioner type menu whereby practitioner data points displayable upon the interactive map are filterable by discipline, category, or type.

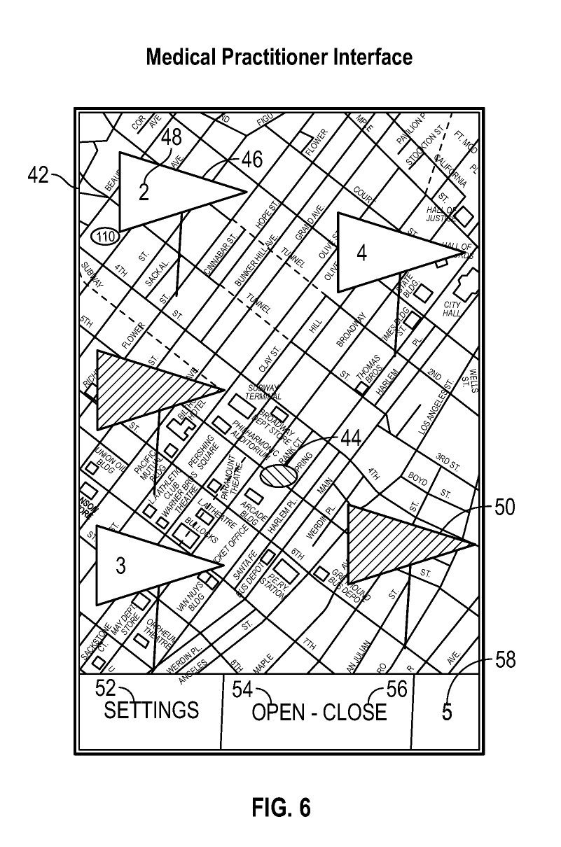

FIG. 6 is an example embodiment of a screen shot showing practitioner side GUI wherein an interactive map is displayable with alternative practitioner data points, and their associated availability and first order information is shown.

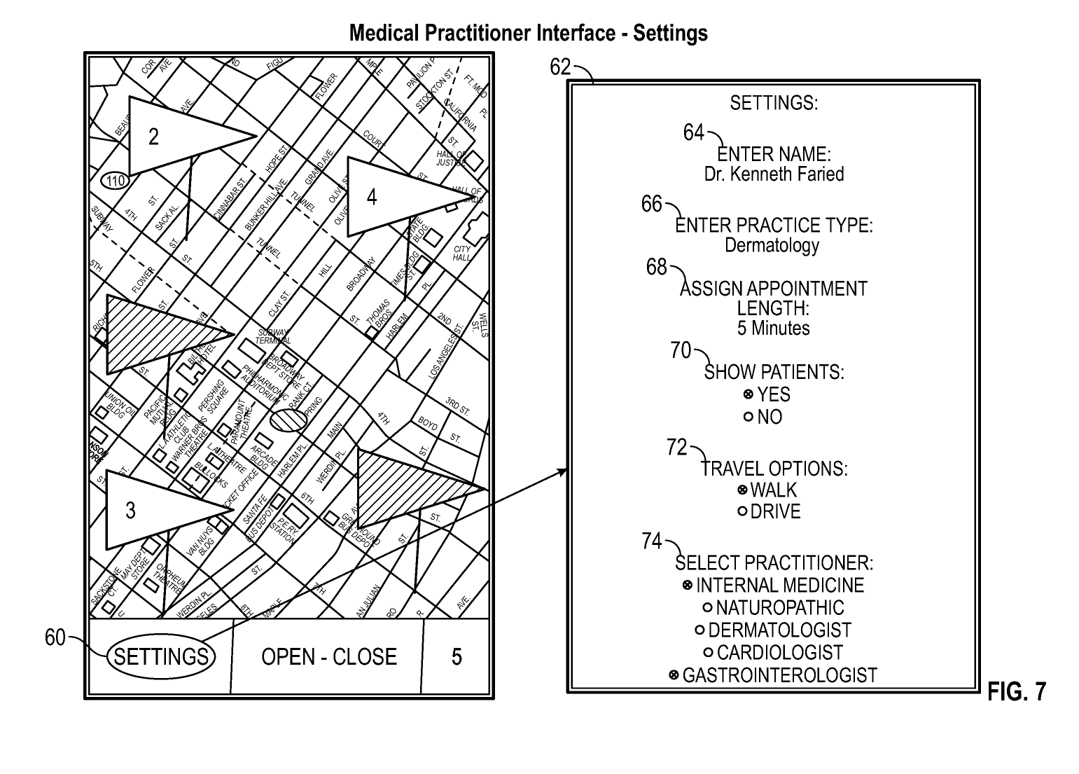

FIG. 7 is an example embodiment of a screen shot showing a settings window interface wherein a practitioner may update information displayable as part of said practitioner's data point and select additional practitioner types to display on the interactive map as well as select and hide relevant patient positions on the interactive map.

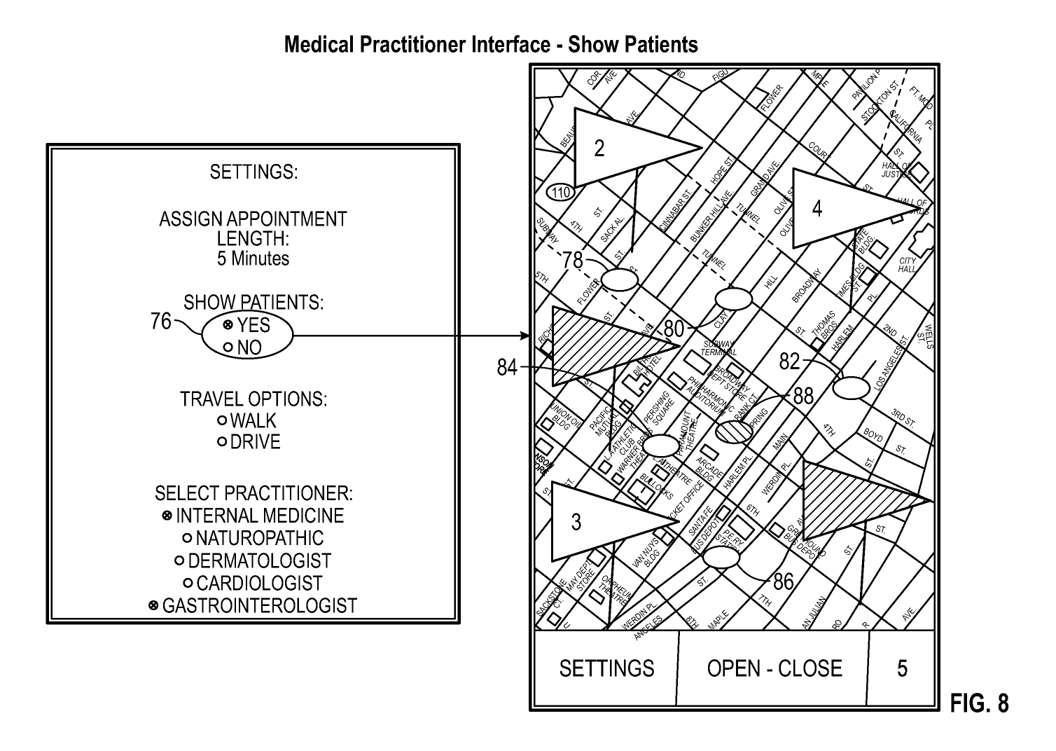

FIG. 8 is an example embodiment of a screen shot showing relevant patient positions upon the interactive map, whereby the instant practitioner may be directed to a particular patient in said practitioner's queue for an in-person visit by selecting any one of the displayed patient positions.

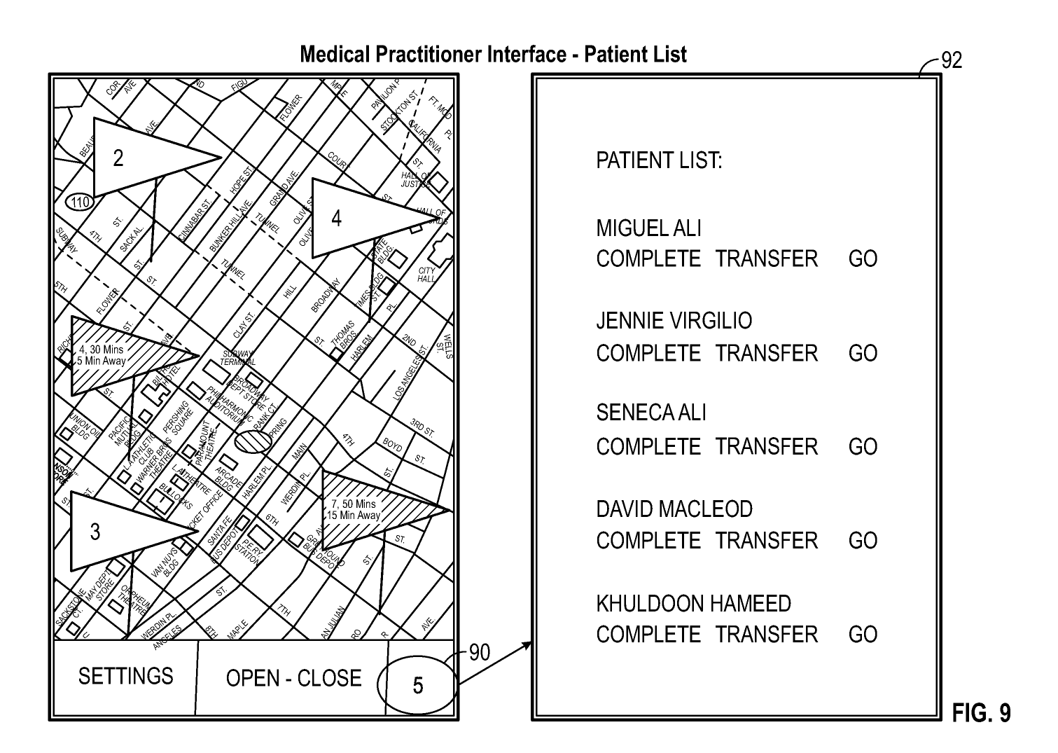

FIG. 9 is an example embodiment of a screen shot showing a practitioner's queue wherein patient names are listed with associated command prompts; a “Complete” link to complete a patient appointment; a “Transfer” link to effect transfer or referral of the patient to another available practitioner selectable through the GUI; and a “Go” link, selectable to route the practitioner to that particular patient.

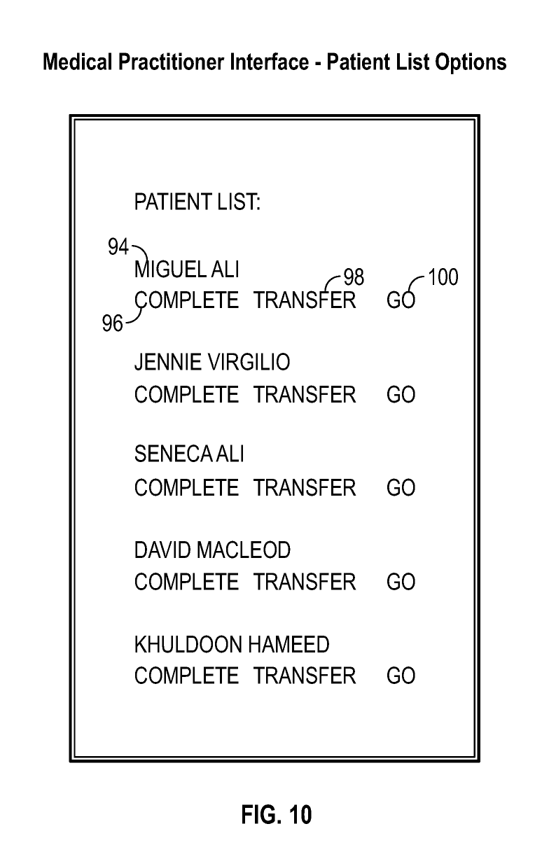

FIG. 10 is an example embodiment of a screen shot showing details of the practitioner queue with patients occupying appointment slots.

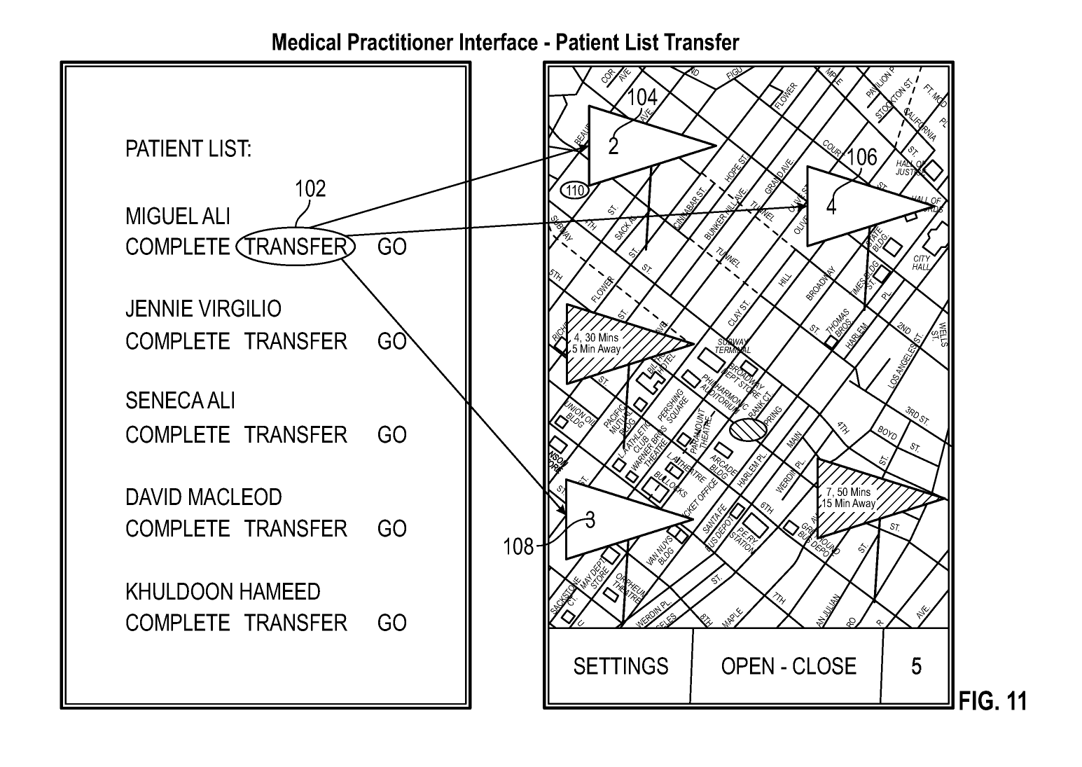

FIG. 11 is an example embodiment of a screen shot showing selection of a patient from the practitioner queue to transfer to available practitioners.

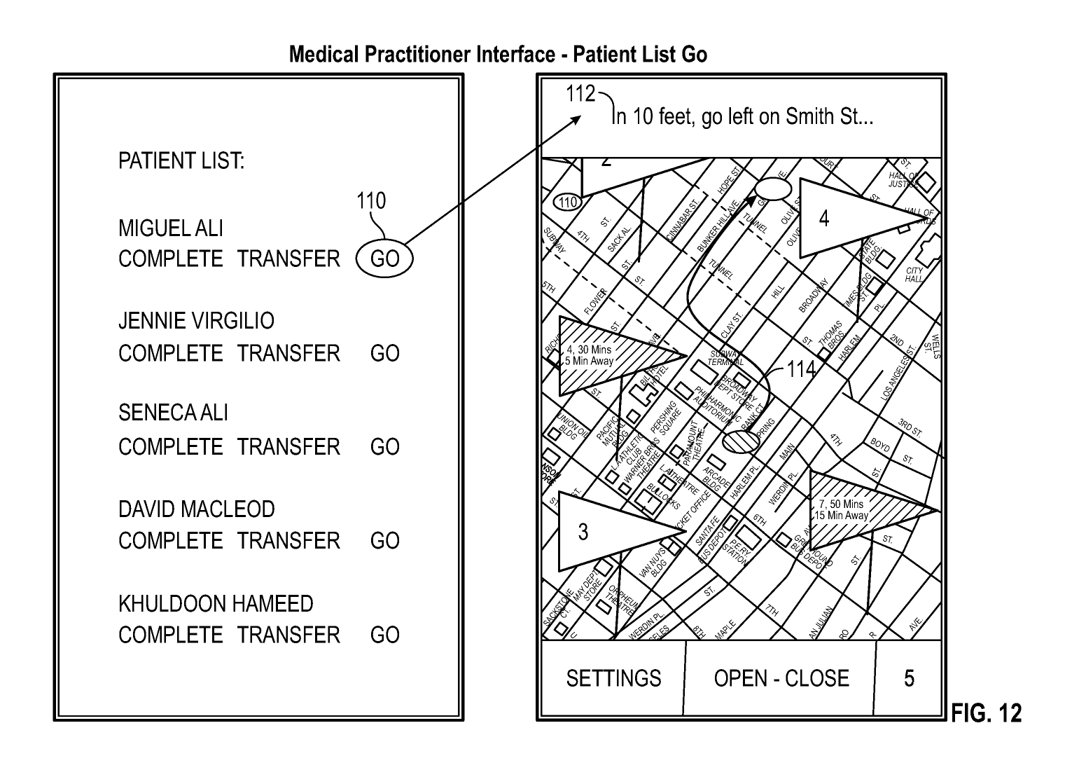

FIG. 12 is an example embodiment of a screen shot showing practitioner selection of the “Go” link to route said practitioner to a particular patient selected from the queue.

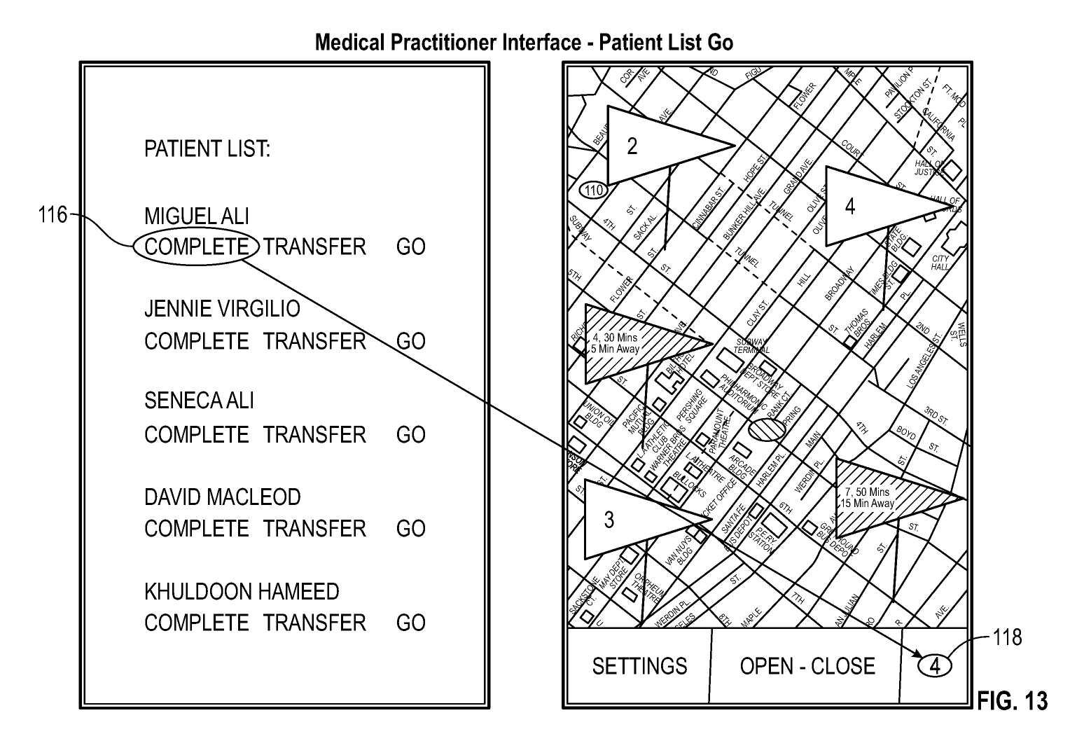

FIG. 13 is an example embodiment of a screen shot showing completion of a patient appointment whereby the practitioner patient number is automatically updated on the practitioner GUI.

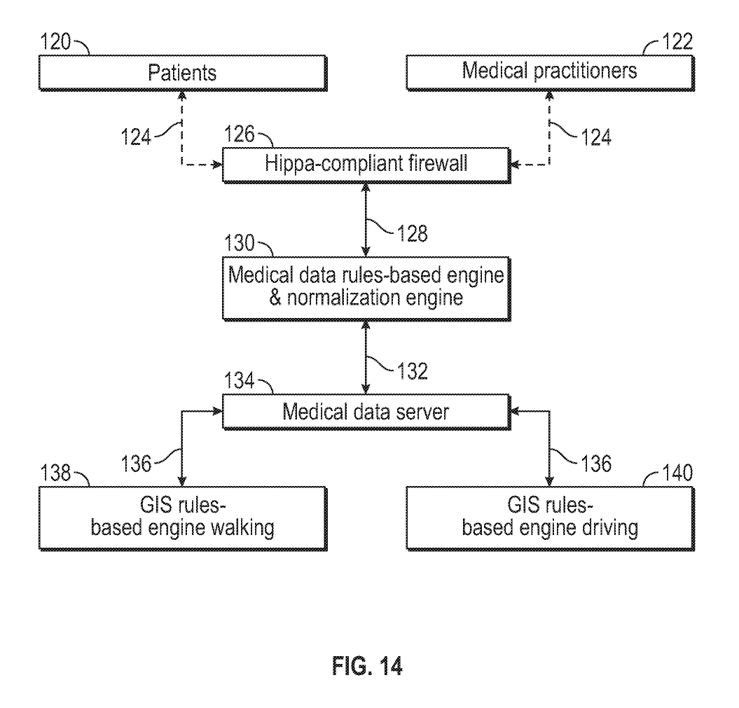

FIG. 14 is a flow diagram illustrating an example embodiment of intermediary to steps comprising data flow through the network across a HIPPA-compliant firewall, a central server, and a Global Positioning System.

### DETAILED DESCRIPTION OF THE DRAWINGS

Referring to FIGS. 1 through 14 an example embodiment of the present method for practitioner network and appointment management system is illustrated.

The present method for practitioner network and appointment management system has been devised to enable end user selection of available medical practitioners determined geographically proximal said end user, said practitioners selectable for appointment scheduling through a graphical user interface (“GUI”) operable by means of a peripheral device (or wireless communication device (“WCD”), as case may be) whereby a practitioner's appointment schedule and availability is updateable across a secure user network.

The terms “peripheral device” and “WCD”, as used herein throughout, are taken to include all computing devices connectable over Internet protocol with a central server, thereby communicable and operable to display updatable information for interaction with an end user. The terms “peripheral device”, “peripheral computing device”, and “WCD” will be used synonymously herein.

The term “central server”, as used herein throughout, is taken to include any centralized distribution of information between end users networked together by means of the present method, and should not be taken to restrict embodiments of the present method to steps wherein information is distributed from a single such central server operating as a standalone computing device. Indeed, the “central server”, as used herein, is taken to include decentralized hubs and points of presence whereby data is distributed and communicable to end users as if said data were managed at a central source. Thus end user information exchanged through the network from a single peripheral device may constitute said central server. The concept is that a single data point is distributed to all users in the network when said single data point is introduced into the network at any participating peripheral device or WCD.

The present method, therefore, is operable upon a WCD to present a GUI to an end user when an application, downloaded to the WCD, is opened. The GUI presents as a selectable menu and a home screen 2 (see FIG. 1), whereon an interactive map, operable by use of a touch screen on a WCD, or by cursor selection (with a computer-aided display control, a “mouse”, for example) on other peripheral or other computing device, enables display of practitioner data points 6 for selection, as desired, and entry of a data input to effect an appointment update.

Practitioner data points 6 display upon the interactive map indicative of relevant location of each practitioner networked into the system, said practitioners displayable by scalable proximity to the end user. Practitioners may be filtered for display by type when selection of a practitioner type menu prompt 10, displayable upon a home screen 2 of the GUI when opened initially on a WCD or computing device, is effected. Practitioners displayed are therefore controlled by categories selectable as a practitioner type, such as, for example, dermatologist, general practitioner, ophthalmologist, urologist, chiropractor, and other such appropriate category as effective to sort practitioners participating in the network by area of practice or specialty, as case may be (see FIG. 5 for example). The interactive map is scalable, whereby the end user is enabled to determine most proximal practitioners of a selected category of medical practice, whereby the end user may locate the most proximal, preferred, or most suitable practitioner from the GUI.

Practitioners display as data points controllable by action through a practitioner side of the network, as detailed hereinbelow. These data points are determined to include relevant visual cues whereby an end user is apprised of the availability of each practitioner's schedule. Such visual cues may include, for example, color change indicating no availability, moderate availability, and availability. Other visual cues are contemplated as part of the invention, such as a percentage availability applied to mornings and afternoons displayable as segments of days in graphical or other form. A time to next available appointment may also be displayed. Thus an end user is enabled selection of an appropriate practitioner depending on proximity, specialty, and availability.

The present method includes use of a Global Positioning System (“GPS”) whereby an end user's present location is determinable and directions to selected practitioners are enabled by actionable display presented through the GUI. Once selected, and data input is entered to effect an appointment update (whereby the user is entered into the appointment schedule of the relevant practitioner), directions to the practitioner from the end user's present location may be effected whereby the WCD routes the end user to the practitioner by foot or vehicle, as case may be.

An appointment update is effective across the network, and the relevant practitioner's schedule is thus displayable with the data input entered that effected the appointment update for all end users and practitioners. Depending on the user type (i.e. end user versus practitioner) the information displayed in a practitioner schedule may be different, with patient sensitive and private data secured confidentially and displayable only to a particular practitioner (and, in most cases, only the practitioner whose appointment schedule was updated).

It is further contemplated as part of the present invention that payment solutions may be effected between parties once an appointment has been completed. Relevant payment information (such as, for example, credit card data or other payment data) may be storable in memory whereby payment may be effected by a user as desired. Relevant transfer of insurance information pertaining to the patient and practitioner may also be automated. Thus a patient is enabled to make payment effected by the present method directly through the participating peripheral device, for a particular appointment and/or service, whereby the practitioner is enabled receipt of required funds and relevant insurance billing information is immediately transferrable.

Use of the GUI and orchestration of the present method by an end user to select a practitioner and update an appointment schedule by relevant data input will now be described.

FIG. 1 shows an example embodiment of a home screen 2 of the GUI operable by an end user to select a desired practitioner and engender an appointment. The home screen 2 includes practitioner data points 6 and the end user position 4 displayable as part of an interactive, scalable map, and a practitioner type menu prompt 10 and the GPS activation links “walk” 12 and “drive” 14. The practitioner data points are here shown as flags 6, however additional means of visually signaling the practitioner data points are contemplated as part of this method, the important feature being visibly distinguishable location of selectable data points upon an interactive map. Availability in this example embodiment is illustrated by a white flag 6. Lack of availability is signaled by a black flag. Thus a user is enabled rapid discernment between available practitioners and unavailable practitioners.

Additional first order information is displayable in each practitioner data point. As shown in FIG. 2, a practitioner data point 16 may include additional first order information such as the practitioner name 18, the time of the next available appointment 20, an availability index (the number of patients in the current queue 22, the approximate waiting time 24), and the estimated travel time 26 to the practitioner. Additional information may be displayed as part of the practitioner data point and, alternatively, first order information (including a user rating, for example) or second order information may be displayable when a user hovers a cursor over a particular practitioner data point or when a data point is selected.

Selection of a practitioner data point upon the interactive map may automate input of end user data into the practitioner queue, whereby an available appointment slot is filled. Automation of end user data input into the practitioner queue may automate transfer of end user information into the practitioner queue for administration on the practitioner side, said end user information including, for example, relevant insurance information, patient biometric data, payment information, and other data. End user information input may likewise enable cross-sharing of patient medical information provided by another service enabling electronic transfer of confidential medical files and data relevant to the particular end user.

As shown in FIG. 3, selection of a practitioner data point manifests as a visual cue by altering the appearance of the data point 28 (shown here as a dotted perimeter bounding the flag, but additional or other visual cues are contemplated as part of this invention) and a confirmation box 30 opens whereby end user confirmation is required to effect data input and effect update of the appointment schedule. In this example embodiment depicted as FIG. 3, selection of the practitioner data point opened end user confirmation box 30, whereby transfer of data and update of appointment schedule across the user network is effective by selection of the confirmation box 30. No additional data input is necessary to effect the appointment update nor entry of additional end user data to apprise the practitioner of patient data, the relevant data automatically transferred to the practitioner queue from cached data storable in the end user WCD (or accessible through a confidential cloud or other peripheral) as will be described subsequently.

FIG. 4 illustrates an example embodiment of a route 36 superimposed upon the interactive map, by which route 36 the end user is directable to reach the selected practitioner from said end user's current location. A direction field 34 may also indicate text-based directions. The route and text-based directions are savable to memory whereby closure of the application running the present method previous sensed completion of the route will not delete the route, rather reopening the application upon a relevant WCD will update the route depending on the end user's sensed present location.

FIG. 5 illustrates an example embodiment of the practitioner type menu prompt selected 38, whereby a practitioner type menu 40 displays, by which practitioner data points displayable upon the interactive map are filterable by practice and/or specialty. In the present example illustrated in FIG. 5, “DERMATOLOGIST” is selected whereby five practitioners of that type are displayed proximal the end user current location, which end user current location is shown as an ovoid dot in the center of the interactive map.

FIG. 6 illustrates an example embodiment of a practitioner view home screen 42 wherein an interactive map is displayable as part of the practitioner side of the network. In this example embodiment the practitioner's location is shown as a dot 44 centrally disposed upon the interactive map and proximal practitioners are displayable as data points 48 indicative of said proximal practitioner locations. Practitioner data points are displayable with visual cues as to current availability to see additional patients, whereby rapid referrals are effective should a practitioner using the network deem referral to another practitioner appropriate for a particular patient.

In the present example embodiment shown in FIG. 6, white flags indicate a practitioner has availability to see more patients. The large integer in each white flag represents the number of patients in each relevant practitioner's queue. Black flags represent practitioners who have no availability at the time the interactive map was last refreshed. An “open” select link 54 and a “close” select link 56 are displayed whereby practitioner selection of either link 54, 56 controls display of said practitioner's own data point whereby said practitioner may signal availability or unavailability to other users in the network. The number of patients 58 in a practitioner's queue is controlled by the application, whereby available slots are fillable by end user selection participating in the network and updateable in real time.

A “settings” select link 52 enables a practitioner to open a settings window interface 62 and enter relevant practitioner data therein. An example embodiment of the settings window interface 62 subsequent effect of the settings select link selected 60 is shown in FIG. 7, wherein a practitioner is enabled control of default settings including practitioner name (“Enter Name” field 64), specialty or discipline (“Enter Practice Type” field 66), length of each appointment slot (“Assign Appointment Length” field 68), enable or disable display of number of patients in queue (“Show Patients” field 70), enable GPS mapping (“Travel Options” field 72), control display of other practitioner types (“Select Practitioner” field 74).

The length of each appointment slot data input (in minutes, or other time unit) controls the calculation of the time to availability for any practitioner, whereby the application multiplies the length of each appointment slot by the number of patients in the queue, whereby a time to availability is calculable. Time to first available slot may also be enabled, whereby appointments made into the future and first vacant slots are determinable.

Selection of the “Show Patients” field 70 to show patients reveals patient position on the interactive map, whereby a practitioner is apprised of patient positions. A patient traveling to the practitioner, therefore, is locatable upon the interactive map when a practitioner selects the “Show Patient” field 70 in the affirmative. As shown in FIG. 8, selection of the “yes” 76 option controlling the “Show Patient” filed 70 reveals patients 78, 80, 82, 84, 86 in proximal position relative the practitioner 88. Selection of any one of these patients 78, 80, 82, 84, 86, may route the practitioner to said any one patient for an in-person visit if preferable.

A practitioner is enabled to refer patients, and transfer patients into other practitioner queues. FIGS. 9 and 10 illustrate an example embodiment of the number of patients link selected 90 whereby the practitioner's queue 92 displays. Patient names 94 currently occupying appointment slots in the practitioner queue are displayable (along with additional information, such as insurance information, biometric data, billing or payment information, among other patient information), and selection of any one of the patient names 94 enables the practitioner to confirm transferring the particular selected patient to another practitioner participating in the network. In this example embodiment, command prompts “complete” link 96, “transfer” link 98 and “go” link 100 enable the practitioner to complete the appointment, transfer the patient to an alternate practitioner available participating in the network, or generate a route and directions to the particular patient on the interactive map respectively, whereby the patient's current location is made visible on the interactive map relative the practitioner's location. FIG. 11 illustrates an example embodiment of effecting a transfer of a patient out of a practitioner's queue into another practitioner's queue by selection of the “transfer” link 102 and subsequently selecting an available practitioner 104, 106, 108 of selected type displayed upon the interactive map.

FIG. 12 illustrates an example embodiment of routing a practitioner to a particular patient by action of the “go” link selected 110 whereby a practitioner is routed with directions to a particular patient and practitioner in-person visit to a patient is enabled. Thus house calls, or other patient visitations are enabled, whereby the practitioner is enabled to go to the patient rather than the patient come to the practitioner, as desired. Video-conferencing and/or telecommunication and/or peer-to-peer communication with selected patients is likewise contemplated as part of these features of the present method.

FIG. 13 illustrates an example embodiment of action of the “compete” link selected 116 updating the number of patients 118 in the practitioner queue. Patient number is updated system wide whereby the practitioner data point displayable upon each user (end user and practitioner) interactive map is updated accordingly. Additionally, the practitioner's time to availability is updated as well.

FIG. 14 shows a flow diagram connecting patients 120 in network 124 with practitioners 122 through a HIPPA-compliant firewall 126, whereby all medical transactions and data transfer in the network is maintained confidential. A central server 134 is disposed in operational communication with a Global Positioning System 138, 140 to control sensed user positions relative each sensed current location of each user.

What is claimed is:

1. A practitioner network and appointment management system runnable upon a peripheral computing device to connect end users and practitioners in a secure network, said system having an end user side and a practitioner side, whereby the end user side enables end user selection of available practitioners displayable on an interactive map, said practitioners determinable to be geographically proximal said end user, said practitioners selectable for appointment scheduling through a GUI displayable upon the peripheral computing device, and the practitioner side enables practitioners access to automatically populated patient data by selection of patients entered into and actively occupying slots in an updateable practitioner queue, whereby an appointment schedule and practitioner availability is continuously updateable in real time across the secure network between end users and practitioners.

2. The practitioner network and appointment management system of claim 1 wherein the end user side further enables display of practitioner data points having visual cues significant of practitioner availability.

3. The practitioner network and appointment management system of claim 2 wherein the end user side further enables display of first order data associated with each of the practitioner data points, said first order data including practitioner name, medical discipline or specialty, number of patients active in the relevant practitioner queue, and a computed time to availability.

4. The practitioner network and appointment management system of claim 3 wherein the time to availability is computed by multiplying the number of active slots in the practitioner queue by an associated length of appointment slot data.

5. The practitioner network and appointment management system of claim 4 wherein the end user side comprises a filter to control practitioners displayed by selection of a practitioner type whereby end users can constrain practitioners displayable on the interactive map sorted by medial discipline and specialty.

6. The practitioner network and appointment management system of claim 5 wherein the first order data displayable on the end user side further includes a travel time to practitioner location determined from a current end user location.

7. The practitioner network and appointment management system of claim 6 wherein the end user side displays travel directions to a selected practitioner.

8. The practitioner network and appointment management system of claim 7 wherein the practitioner side enables display of patient data of an active end user in the practitioner queue by selection of a patient name displayed in the slots of the practitioner queue.

9. The practitioner network and appointment management system of claim 8 wherein the patient data includes insurance information and a medical history of the selected end user, said patient data automatically retrievable over network by secure protocol for display on the practitioner side when the patient name is selected.

10. The practitioner network and appointment management system of claim 9 wherein the practitioner side further enables transfer of end users between practitioners by practitioner selection of a targeted end user in the practitioner queue and effecting a transfer of said selected end user to another practitioner participating in the network by selection of another practitioner displayed on the practitioner side interactive map whereby all relevant patient data is automatically transferrable.

11. The practitioner network and appointment management system of claim 10 wherein automated payment between end users and practitioners is enabled and effected by end user confirmation of a balance upon said end user's peripheral computing device, whereby financial transactions for medical services are transactional and automatable via the secure network.

12. A practitioner network and appointment management system runnable upon a peripheral computing device to connect end users and practitioners in secure network, said system having an end user side and a practitioner side, said system comprising:
generation and presentation of an interactive map as part of a GUI, said interactive map scalable between a minimum area and a maximum area for end user and practitioner display;
on an end user peripheral device:
enabling display of practitioner data points upon the interactive map, each of said practitioner data points demarking locations of particular practitioners connected in network relative a determined current location of an active end user, said practitioner data points including first order data selectable by each said end user upon the interactive map;
enabling filtering of practitioner data points by categories of practitioner type whereby end users can delimit display to practitioner data points of a particular practitioner type;
visually signaling cues to end users as to availability of each practitioner associated with each displayed practitioner data point, said visual cues including a computed time to availability;
displaying a practitioner schedule upon selection of an associated practitioner data point;
effecting an appointment update by end user selection upon the practitioner schedule, said appointment update signaled across the network to all participating users;
effecting travel directions to a selected practitioner when an end user selects a particular practitioner data point;
on a practitioner peripheral device:
enabling display of a practitioner location relative other practitioner locations;
enabling display of current end user locations upon the interactive map; and
enabling display of an associated practitioner queue of the practitioner, said practitioner queue listing appointment slots and patient names therein, said patient names selectable to reveal additional information regarding patient data, such as insurance information, biometric data, billing and payment information, and other patient information, said names selectable to enable transfer of a selected patient as a referral to another practitioner;
wherein practitioner availability is determinable on end user peripheral computing devices and end users and practitioners are enabled automated appointment scheduling across network relative said practitioner availability, whereby end user information is automatable to populate patient data on the practitioner side of the network and practitioners are enabled to transfer patients to other practitioners active in the network.

13. The practitioner network and appointment management system of claim 12 further comprising automated payment between end users and practitioners effective by end user confirmation of billing received upon said end user's peripheral computing device, whereby financial transactions for medical services are transactional and automated via the secure network.

14. The practitioner network and appointment management system of claim 13 wherein patient data is automatically transferrable to the practitioner queue from cached data securably storable in the end user peripheral computing device and alternately populated from a remotely networked central server via secure protocol.
